(19) United States
(12) Patent Application Publication
(10) Pub. No.: US 2026/0124427 A1
(43) Pub. Date: May 7, 2026

(54) PROGRESSIVE-IMPLICIT-LEARNING FOR RECOVERING FROM BRAIN DISRUPTION

(71) Applicant: RE-SETS LTD, Hod-HaSaron (IL)

(72) Inventors: Avi Harel, Hod-HaSaron (IL); Yossi CHALAMISH, Kibutz Harel (IL); MOSHE Horev, Tel Aviv (IL)

(73) Assignee: RE-SETS LTD, Hod-HaSaron (IL)

(21) Appl. No.: 19/441,337

(22) Filed: Jan. 6, 2026

Related U.S. Application Data
(63) Continuation-in-part of application No. 18/913,995, filed on Oct. 11, 2024.
Application No. 18/913,995 is a continuation-in-part of application No. 18/242,773, filed on Sep. 6, 2023.
Application No. 18/913,995 is a continuation-in-part of application No. 18/242,852, filed on Sep. 6, 2023.
(60) Provisional application No. 63/414,878, filed on Oct. 10, 2022.
(60) Provisional application No. 63/414,878, filed on Oct. 10, 2022.

(51) Int. Cl. A61M 21/02; G16H 40/67

(52) U.S. Cl. CPC A61M 21/02; G16H 40/67; A61M 2230/06

(57) ABSTRACT

An example of a non-transitory computer readable storage device is disclosed. The storage device may comprise executable instructions that when executed cause a processor at an Individual's-Brain-Disruption-Release Controller (IBDRC) to obtain two or more intervals of a biological-parameter, wherein each interval is associated with a stage of a progressive-implicit-learning, to determine that a current reading from a monitoring device, which is associated with the IBDRC and is configured to monitor the biological-parameter, is in the interval of a current stage of the progressive-implicit-learning and to instruct an individual, who is associated with the IBDRC, to executed an anchoring-ritual (AR).

### CROSS REFERENCE TO RELATED PATENT APPLICATIONS

This is a utility patent application that is being filed in the United States Patent and Trademark Office as a non-provisional application for patent under Title 35 U.S.C. § 100 et seq. and 37 C.F.R. § 1.53(b) and is a continuation-in-part of the United States application for patent that was filed on Oct. 11, 2024, and assigned Ser. No. 18/913,995, which application is:
a continuation-in-part of the United States application for patent that was filed on Sep. 6, 2023, and assigned Ser. No. 18/242,773, which application claims the benefit of the prior filing date under Title 35, U.S.C. § 119(e) of the United States provisional application for patent that was filed on Oct. 10, 2022 and assigned the Ser. No. 63/414,878, and
a continuation-in-part of the United States application for patent that was filed on Sep. 6, 2023, and assigned Ser. No. 18/242,852, which application claims the benefit of the prior filing date under Title 35, U.S.C. § 119(e) of the United States provisional application for patent that was filed on Oct. 10, 2022 and assigned the Ser. No. 63/414,878.

Each of the above-identified applications are herein incorporated by reference in their entirety.

### FIELD OF THE DISCLOSURE

The present disclosure relates to the field of improving the quality of life and the wellbeing of a person.

### BACKGROUND

From time to time a person may suffer from brain disruption such as but not limited to chronical-psychological-stress (CPS), sleep disorder, primary headache, Migraine, etc. In addition a person may suffer from Body-Mind-Disorder (BMD). BMD such as but not limited to Auto-emotion-Disorder, Dementia, etc. Along the disclosure and the claims the term stress can be used as a representative term for any type of brain/mind disruption. The stress can be a result of a trauma that the person suffered in the past. In some cases the stress may involve fears, memory attenuation, physical balance, emotional balance, etc. The effect of stress may be increased along the years. The stress may be associated with the human senses. Senses such as but not limited to smelling, seeing, hearing, tasting, etc.

Usually the brain disruption may interfere with daily life and wellbeing of that person. It may reduce performance of the person and may reduce the quality of life of that person. Common methods for handling brain disruption may comprise medicines, treating the person at a clinic by a psychotherapist, etc. In the long run using medicines may affect the health of that person. It may affect the kidney, the liver, etc. of that person.

Therefore there is a need for a new system and method which will help a person to overcome brain disruption without medicines. Along the present disclosure and the claims the terms chronical-psychological-stress, stress, trauma or brain disruption can be used interchangeably and the term stress can be used as a representative term to this group.

### BRIEF SUMMARY

The needs and the deficiencies that are described above are not intended to limit the scope of the inventive concepts of the present disclosure in any manner. The needs are presented for illustration only. The disclosure is directed to a novel system for progressive treatment of the brain of an individual in order to overcome a brain disruption such as but not limited to stress situation.

An example system for progressive treatment of the brain may comprise a Brain-Disruption-Release-Server (BDRS), and one or more Individual's-Brain-Disruption-Release-System (IBDRS). An example of BDRS may comprise a plurality of Progressive-brain-disruption-release-applications that can be downloaded to one or more IBDRS in order to treat individuals that suffer from Brain-Disruption.

An example of an IBDRS may comprise, among other elements, an Individual's Brain-Disruption-Release Controller (IBDRC) and a monitoring device that is capable to monitor at least one biological parameter that is related to the brain disruption. A non-limiting example of a monitoring device can be pulse rate monitoring device (PRMD) such as but not limited to a ring “R10” manufactured by COLMI. Another non-limiting example of a monitoring device can be a smart watch such as but not limited to “VIVOACTIVE 5” manufactured by Garmin USA. Those monitoring devices can be used for monitoring the pulse rate, which has high correlation to the level of stress of the individual, for example. Other monitoring devices can measure other biological parameters such as but not limited to Galvanic-skin-response (GSR) or heart-rate-variations (HRV), etc.

An example of IBDRC can be a smart-phone, a laptop, a personal computer, etc. The IBDRC can be configured to download one or more types of Progressive-brain-disruption-release-application (PBDRA) from the BDRS.

The novel progressive treatment for recovering from a brain disruption may comprise few stages. The first stage is the introducing stage, in which a therapist may present the progressive treatment to an individual and get the individual's agreement to participate in the progressive treatment. Then the therapist may collect information that is related to the individual. The information may comprise age, profession, education, family status, type of music that the individual likes, etc.

Next, the therapist may interview the individual in order to determine whether the individual, who suffers from brain disruption, is suitable for the novel treatment. In order to be suitable the individual needs to have at least one period of time, in which the individual did not suffer from the brain disruption. Thus, whether the individual has autobiographic-positive-memory (APM). APM of events or periods in which the individual feels eustress, vividness, etc. If the individual has APM and is willing to participant in the progressive treatment as it was disclosed by the therapist, then the first stage can be continued.

The therapist may ask the individual to describe the individual's weekly schedule of a common week, day by day. The schedule may comprise the awaken hour, dining hours, exercising hours, working hours, resting hours, etc. Further, the therapist may collect information about states that lead the individual into stress. States such as but not limited to sit in a car while someone else is driving it, being in a place with cockroaches, rats, etc.

Then the therapist may lead the individual into relaxation. Leading the individual into relaxation can be done by the therapist that discloses events that are associated with the APM while playing the type of music that the individual likes. The voice of the therapist and the music can be recorded as recorded file number 1 (RecF1). The RscF1 is given to the individual and the individual is requested to exercises RecF1 one or more times during each day while monitoring the pulse rate. At home, the individual may exercise the RecF1 every day during few weeks, one to three weeks, two weeks for example. In addition the individual is requested to write the level of vividness that the individual feels during listening to the RecF1. The level can be between one to five, wherein five represents high vividness.

Last but not the least, the therapist may present the bio monitoring device, the ring or the watch that can measure the pulse rate for example, and explains how to use it. Then, the individual is connected to the monitoring device and the therapist may start playing the RecF1. The individual is requested to indicate when he feels that he is in a state that is related to the APM that is disclosed in RecF1. The indication can be raising a hand, for example. At this point the reading from the monitoring device is stored in the IBDRC. Some example embodiments of the disclosed technique may use neuro waves monitoring device such as but not limited to Muse. Muse is a headband monitoring device that is manufactured by InteraXon a company that is located at Canada.

Each training with the RecF1 is recorded and be stored together with the reading of the monitoring devices (pulse rate, conductivity of the skin, stress, etc.) as well as the reports of the individual about the level of vividness that the individual felt during the training. When the therapist determines that the mind of the individual is ready to do shifting of attention, then the therapist leads the individual into the next step of the progressive treatment. In some example embodiments the therapist may use a neuro waves monitoring device in order to determine when the mind of the individual is ready to do shifting of attention.

In order to save the time of the descripting the APM the individual is requested to executed an anchoring-ritual (AR). An example of AR can be associating the finger with the thumb of the right hand, or to put the right hand on the head, etc. Some example embodiments may use neurofeedback practice in order to generate the vividness via relaxation.

The therapist can persuade the individual that the anchoring-ritual represents the APM. Thus, the AR can reduce the stress of that individual by shifting his attention from the current situation of stress into positive memory that generates vividness. The therapist may give a second recording file to the individual (RecF2). The RecF2 comprises the voice of the therapist that leads the individual into vividness via relaxation. At this point of time the individual is requested to execute the AR. Then, the therapist may ask the individual to exercise the RecF2 and the AR, at home when the individual is relaxed and in good time. By using the AR the individual activates his auto biography positive memory and bring past-emotional-experience-memory (PEEM) into the present. Thus, by using the AR the individual shift the attention of his mind from the present to the past. Persuading the Individual can be done by Hypnosis, meditation or just by talking.

The one or more monitoring devices are configured to report every few tens of seconds, 30 seconds for example, to the IBDRC the value of the one or more bio parameters of the individual. The bio parameters can be pulse rate, level of stress, conductivity of the skin, etc . . . Along the present disclosure and the claims the parameter pulse-rate can be used as a representative term to any biological parameter.

During training at home, the individual is requested, to report the level of vividness that he felt during executing the AR. This report is done on the IBDRC and be transferred to the server. When the therapist determines that the AR is working while the individual is in good condition, then the therapist moves to the next phase of the progressive training.

The next phase can be referred as implicit learning of that individual. The implicit learning can be executed in stages. Some example embodiments may use three stages, other embodiments my use seven stages, etc. We found the five stages give good results. The stages can be defined by a scale of monitoring values. An example PBDRA can be configured to present a scale of five stages starting with the pulse rate of 60 pulses-per-minute (PPM) for example. The following stage can start with 70 PPM, the next will start with 80 PPM, then 87 PPM and the last one will start with 95 PPM, for example. The PBDRA can be configured to verify that the lowest measured pulse rate of that individual is in between the first threshold (60 PPM) and the following threshold (70 PPM). If not the PBDRA is configured to amend the scale.

At this point the implicit-bio-learning can be started. During the first stage the application, when it determines that the current pulse-rate is in the first interval (between 60 to 70 PPM, for example) is configured to instruct the individual to execute the AR and to report whether the AR was effective and the application store the pulse rate that was measured during the AR. The individual is requested to train this stage during several days until the application determines that the AR reduces the pulse rate of the individual and the individual reports that the AR improves the individual's vividness. The number of days can be a week or two, for example

During the second stage, if the application determines that the current pulse-rate is in the second interval (between 70 to 80 PPM, for example), then the application is configured to instruct the individual to execute the AR and to report whether the AR was effective. The application may store the pulse rate as well as the feeling of the individual that was measured during the AR. The individual is requested to train this stage during several days until the application determines that the AR reduces the pulse rate of the individual and the individual reports that the AR improves his vividness. The number of days can be a week or two, for example.

In a similar way the treatment may proceed up to the highest stage in which the measured pulse-rate is above 95 PPM, for example. Then, the individual is requested to execute the AR and to report whether the AR was effective. Next the individual is requested to train this stage during several days until the application determines that the AR reduces the pulse rate of the individual and the individual reports that the AR improves his vividness. At this point of time the treatment is completed and the application can be configured to execute the maintenance section of the treatment.

The foregoing summary is not intended to summarize each potential embodiment or every aspect of the present invention, and other features and advantages of the present invention will become apparent upon reading the following detailed description of the embodiments with the accompanying drawings and appended claims.

Further, although specific embodiments are described in detail to illustrate the inventive concepts to a person skilled in the art, such embodiments can be modified to various modifications and alternative forms. Accordingly, the figures and written description are not intended to limit the scope of the inventive concepts in any manner.

Other objects, features, and advantages of the present invention will become apparent upon reading the following detailed description of the embodiments with the accompanying drawings and appended claims.

### BRIEF DESCRIPTION OF THE DRAWINGS

Examples of embodiments of the present disclosure will be understood and appreciated more fully from the following detailed description, taken in conjunction with the drawings in which:

FIG. 1 illustrates a simplified block diagram with relevant elements of an example embodiment of an environment for progressive treatment of the brain of an individual in order to overcome a brain disruption;

FIG. 2 schematically illustrates a flowchart showing relevant processes that can be implemented by an application executing over a therapist computing device for handling a new individual;

FIG. 3 schematically illustrates a flowchart showing relevant processes that can be implemented by an IBDRC during a progressive-implicit-learning of an individual; and

FIG. 4 schematically illustrates a flowchart showing relevant processes that can be implemented by an IBDRC during a executing a maintenance application.

### DESCRIPTION OF EMBODIMENTS

Turning now to the figures in which like numerals represent like elements throughout the several figures, of embodiments of the present disclosure that are described. For convenience, only some elements of the same group may be labeled with numerals. The purpose of the drawings is to describe exemplary embodiments and is not for production purpose. Therefore features shown in the figures were chosen only for convenience and clarity of understanding.

In the following description, for purposes of explanation, numerous specific details are set forth in order to provide a thorough understanding of the invention. It will be apparent, however, to one skilled in the art that the invention may be practiced without these specific details. In other instances, structure and devices are shown in block diagram form in order to avoid obscuring the invention. Moreover, the language used in this disclosure has been principally selected for readability and instructional purposes, and may not have been selected to delineate or circumscribe the inventive subject matter, resort to the claims being necessary to determine such inventive subject matter.

Reference in the specification to “one embodiment” or to “an embodiment” means that a particular feature, structure, or characteristic described in connection with the embodiments is included in at least one embodiment of the invention, and multiple references to “one embodiment” or “an embodiment” should not be understood as necessarily all referring to the same embodiment.

In this specification, these implementations, or any other form that the invention may take, may be referred to as techniques. In general, the order of the steps of disclosed processes may be altered within the scope of the invention. Unless stated otherwise, a component such as a processor, a server, a control unit or a non-transitory computer readable storage device described as being configured to perform a task may be implemented as a general component that is temporarily configured to perform the task at a given time or a specific component that is manufactured to perform the task. As used herein, the term ‘processor’ can refer to a computer such as but not limited to Intel NUC, wherein NUC stands for Next-Unit-of-Computing or “Amazon EC2 A1 Instances” or “Amazon EC2 P3 Instances”, which are maintained by Amazon Crop USA, for example.

Although some of the following description is written in terms that relate to software or firmware, embodiments may implement the features and functionality described herein in software as desired, including any combination of cloud resources, 3^{rd} party virtual machines, application-program-interface (API), etc.

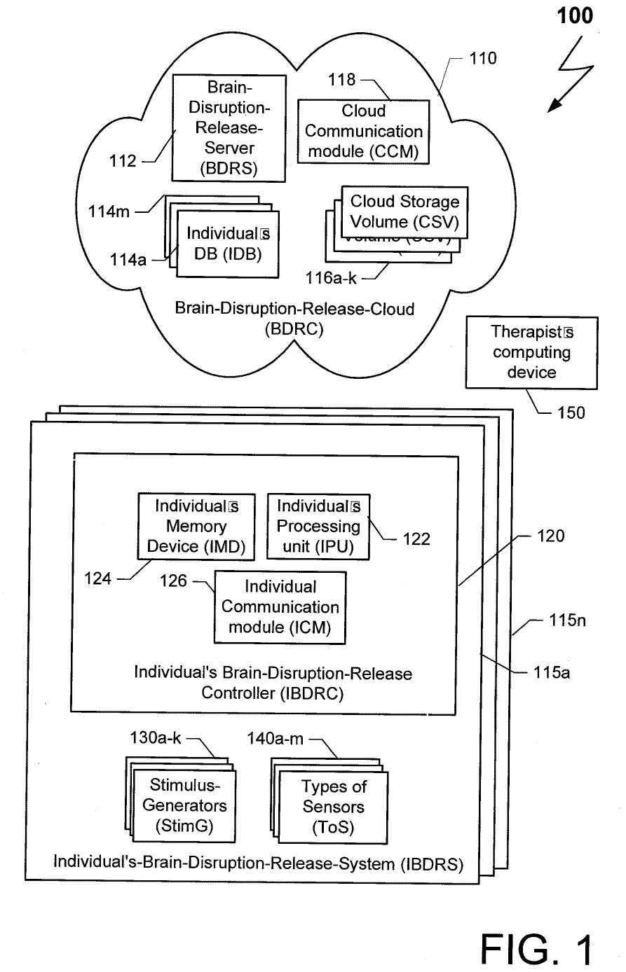

FIG. 1 depicts a block diagram with relevant elements of an example of a Brain-Disruption-Release-Environment (BDRE) 100 in which systems and/or methods, described herein, can be implemented. BDRE 100 may comprise a Brain-Disruption-Release-Cloud (BDRC) 110, one or more Individual's-Brain-Disruption-Release-System (IBDRS) 115a-n and a therapist computing device (TCD) 150. Each IBDRS 115a-n may comprise an individual-Brain-Disruption-Release-Controller (IBDRC) 120, one or more stimulus-generators (StimG) 130a-k and one or more types of sensors (ToS) 140a-m. Wherein at least one ToS 140 is configured to measure the heart pule rate of an individual. One or more of elements of BDRE 100 can comprise one or more processors that are embedded in one or more computers. The computer can be Intel NUC, wherein NUC stands for Next-Unit-of-Computing or “Amazon EC2 A1 Instances” or “Amazon EC2 P3 Instances”, which are maintained by Amazon Crop USA, for example.

An example of a BDRC 110 may comprise a Brain-Disruption-Release-Server (BDRS) 112, one or more cloud-storage-volume (CSV) 116a-k; a cloud-communication-module (CCM) 118 and one or more Individual's DB (IDB) 114a-m. Wherein DB stands for database. An example of BDRS 112 can be configured to manage the operation of BDRC 110. It can be configured to communicate, via CCM 118, with one or more sensors manufactures, stimulus generators manufactures, health websites, etc. In order to collect information that may be needed during the operation of the BDRE 100. The collected information can be stored in CSV 116a-k.

The communication via CCM 118 can be implemented over a packet switch network such as but not limited to an Internet Protocol (IP) network. The IP packets can be transferred over a cellular network, a mobile network, a radio network and or over Public Switched Telephone Network (PSTN). Those networks are well known to a person having ordinary skill in the art and will not be further disclosed.

CSV 116a-k can be one or more non-transitory computer readable storage devices that are configured to store software code (applications) to be executed by BDRS 112 and one or more IBDRS 115a-n. In addition CSV 116a-k can store data and software code to be used by one or more therapists computing device (TCD) 150. The therapist data may comprise pictures, video files, music files, etc. that can be used during a treatment.

The individual DB (IDB) 114a-m can be one or more non-transitory computer readable storage devices that are configured to store data that is related to the individuals. Each individual has a section in the IDB 114a-m. An individual's section may comprise personal data of the individual, information about the individual brain disruption, for example stress, reasons that activate the disruption, current stage of the treatment, relevant events, video session that influence the individual, music files that the individual likes, autobiographic-positive-memory (APM) of that individual, recordings of previous meetings, etc.

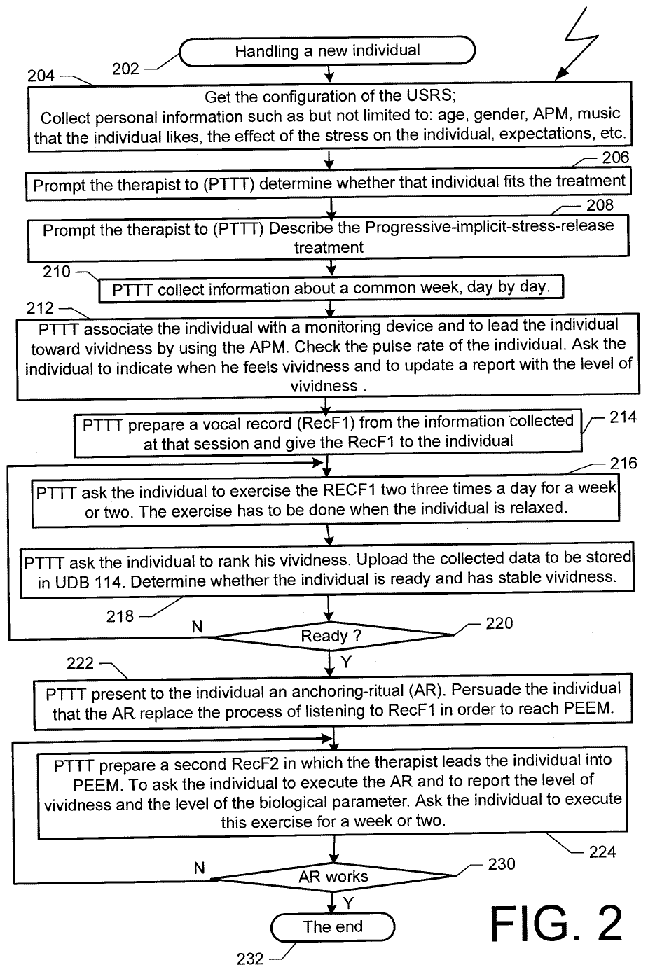

An example of BDRS 112 can be configured to manage the operation of the BDRC 110. It can be configured to communicate, via CCM 118, with one or more sensors manufactures, stimulus generators manufactures, health websites, etc. In order to collect information that may be needed during the operation of the Brain-Disruption-Release-Environment (BDRE) 100. The collected information can be stored in CSV 116a-k. In some example embodiments of BDRC 110, BDRS 112 can be configured to process the information that is collected during treating an individual and based on the collected information it may offer one or more treatments for the following one or more meetings with that individual. More information on BDRS 112 is disclosed below in conjunction with FIG. 2 to FIG. 4.

An example of the therapist's computing device 150 can be a laptop, a personal computer, a hand held computer, a Personal-digital-assistant (PDA), etc. The therapist's computing device 150 can be configured to communicate with the BDRS 112 in order to download one or more applications to be used during a current treatment. More information about the therapist's computing device is disclosed below in conjunction with FIG. 2 to FIG. 4.

An example of an IBDRS 115a-n may comprise, among other elements, an Individual's Brain-Disruption-Release Controller (IBDRC) 120, one or more Stimulus-Generators (StimG) 130a-k and one or more Type of Sensors (ToS) 140a-m. A non-limiting example of a ToS 140a-m can be heart-pulse-rate monitoring device (PRMD) such as but not limited to a ring “R10” manufactured by COLMI. Another non-limiting example of a monitoring device can be a smart watch such as but not limited to “VIVOACTIVE 5” manufactured by Garmin USA.

Example of StimG 130a-k can be smell-generators, music-generators, light-generators, picture-generator, video-generator, speech-generator, tone (sound) generator, self-hypnosis generator, relaxation generator, VR system, etc. An example of a tone generator 130a-k can be Online-Tone-Generator by Tomasz Szynalski or Frequency-Sound-Generator by Google, etc. An example of smell generator 130a-k can be “Ninu-smart-perfume” Another example of smell generator 130a-k can be SmX1 manufactured by SensoryCo California, etc. A slide generator or video generator 130a-k can be associated with the display of the IBDRC 120 and may deliver pictures. The pictures can be delivered from the individual-memory-device (IMD) 124 or from the IDB 114a-m or from a public database, An example of individual-Brain-Disruption-Release-Controller (IBDRC) 120 can be a smartphone, laptop, a hand held computer, a Personal-digital-assistant (PDA), etc. The IBDRC 120 can be configured to communicate with the BDRS 112, via ICM 126 and CCM 118, in order to download one or more applications to be used during a current treatment. An example of IBDRC 120 is configured to communicate with BDRS 112 and the therapist computing device 150 via the individual's communication module (ICM) 126. The communication can be implemented over a packet switch network such as but not limited to an Internet Protocol (IP) network. The IP packets can be transferred over a cellular network, a mobile network, a radio network and or over Public Switched Telephone Network (PSTN). More information about the IBDRC 120 is disclosed below in conjunction with FIG. 2 to FIG. 4.

FIG. 2 schematically illustrates a flowchart showing relevant processes of method 200 that can be implemented for handling a new individual. This process can be executed as an application that is running over the therapist computing device 150 (FIG. 1). After initiation 202 the application 200 may open a form of a questioner that the therapist needs to fill 204 by interviewing the individual. The form may comprise information related to the status of the individual. The status may comprise: age, gender, music that the individual likes, relevant APM, information about the reasons for stress, when the stress started, does he had period without stress, how the stress affects the individual life, etc. In addition the therapist may ask the individual to describe 204 his expectations from the treatment.

At the end of this meeting 206 the therapist is prompted to determine whether the individual fits the following treatment. If yes, the process proceeds to block 208. If not the individual is released and process 200 terminates. At block 206 the therapist, needs to determine whether at the end of the treatment the individual will feel better than now. In order to be fit to the program the individual needs to have autobiographic-positive-memory (APM). APM of events, periods in which the individual feels eustress, vividness, etc. and is ready to participant in that treatment.

Next, the therapist is prompted 208 to describe the progressive-implicit-stress-release treatment to the individual and get the individual's agreement to participant in the process.

If the individual fits the program, then process 200 can prompt the therapist to (PTTT) collect 210 information regarding the individual's weekly schedule of a common week, day by day. The schedule may comprise the awaken hour, dining hours, exercising hours, working hours, resting hours, etc.

Then in block 212 the therapist is requested to associate the individual with a monitoring device. An example of monitoring device can be heart-pulse-rate monitoring device (PRMD) such as but not limited to a ring “R10” manufactured by COLMI. Another non-limiting example of a monitoring device can be a smart watch such as but not limited to “VIVOACTIVE 5” manufactured by Garmin USA. The monitoring device can be configured to send the pulse rate every few tens of seconds, 20 to 60 seconds, 30 seconds for example.

Further, the therapist may lead 212 the individual toward vividness by using the individual's APM while recording the pulse rate every 30 seconds, for example. The individual is requested to indicate when he feels vividness and to indicate in a table the level of vividness from 0-5, in parallel the appropriate pulse rate is stored by the application. Wherein zero indicates no vividness and 5 indicates high level of vividness. Other example embodiments of the disclosed technique may use other number of levels, 3 levels for example. The therapist can be prompt to prepare 214 a vocal record file, RecF1, from the information collected at that session. Then the therapist may give the RecF1 to the individual and be prompted to ask 216 the individual to exercise the RecF1 two to five times a day per a week or two. The exercises have to be done when the individual is relaxed at a quite location, his home for example.

At the end of each exercise the individual is requested to mark 218 the level of vividness that the individual felt in parallel the pulse rate can be recorded too. Some example embodiment of the disclosed technique may use neuro waves monitoring device in order to obtain an objective indication of the level of vividness of that the individual. This information is uploaded and be stored in the appropriate IDB 114a-m. The therapist can analyzes the stored data and determine whether the individual is ready to proceed.

If 220 the individual is not ready, then process 200 returns to block 216 and the therapist may ask the individual to continue exercising RecF1 for additional week or two. If 220 the therapist determines that the individual is ready and has stable vividness, then process 200 can PTTT present 222 the anchoring-ritual (AR) and to persuade 222 the individual that the anchoring-ritual replace the process of listening to RecF1 in order to reach PEEM. Thus, the AR can leads the individual into vividness by shifting his attention from the current situation into PEEM that indicates vividness. An example of AR can be associating the finger with the thumb of the right hand, or to put the right hand on the head, etc. Persuading and leading the Individual can be done by Hypnosis, meditation or just by talking.

At block 224 the therapist can be prompted to prepare a second vocal file, RecF2. RecF2 may comprise the voice of the therapist that leads the individual into vividness via relaxation by activating the individual's auto biography positive memory and bring a PEEM by executing the AR. Then, the therapist may ask the individual to exercise the AR, according to RecF2, at home when the individual is relaxed and in good time. The individual is requested to execute the AR two/three times a day for few weeks while recording the biological parameter, such as but not limited to pulse rate, during AR. By using the AR the individual activate his auto biography positive memory and bring a PEEM into the present. Thus, by using the AR the individual shift the attention of his mind from the present (stress) to a positive moment in the past.

At the end of the week or two a decision 230 is made whether the AR works and led the individual into vividness. The decision can be made by asking the individual and by reading the values from the individual monitoring device 140a-n by checking an HRV monitor, for example. Checking the HRV monitor delivers an objective indication of the client status and his progress in the treatment. In other example embodiments the monitoring device 140a-n can be configured to automatically transmitting the values of the biological parameter toward the IBDRC 120 (FIG. 1). Monitoring device such as but not limited to ring “R10” manufactured by COLMI, which is configured to measures the pulse rate of the individual. If 230 the individual does not reach vividness, then process 200 returns to block 224 asking the individual to exercises the RecF2 and using the AR for other few weeks.

After few times, 3-5 times for example, that the individual failed to achieve vividness by using the AR, then the individual may be requested to consult with the therapist and process 200 can be terminated 232. If 230, the AR works for at least 3 to 5 following days, which means that by using the AR the individual succeeds to reach vividness. This indicates that the individual is ready to move to the next stage of the progressive treatment and process 200 can be terminated 232.

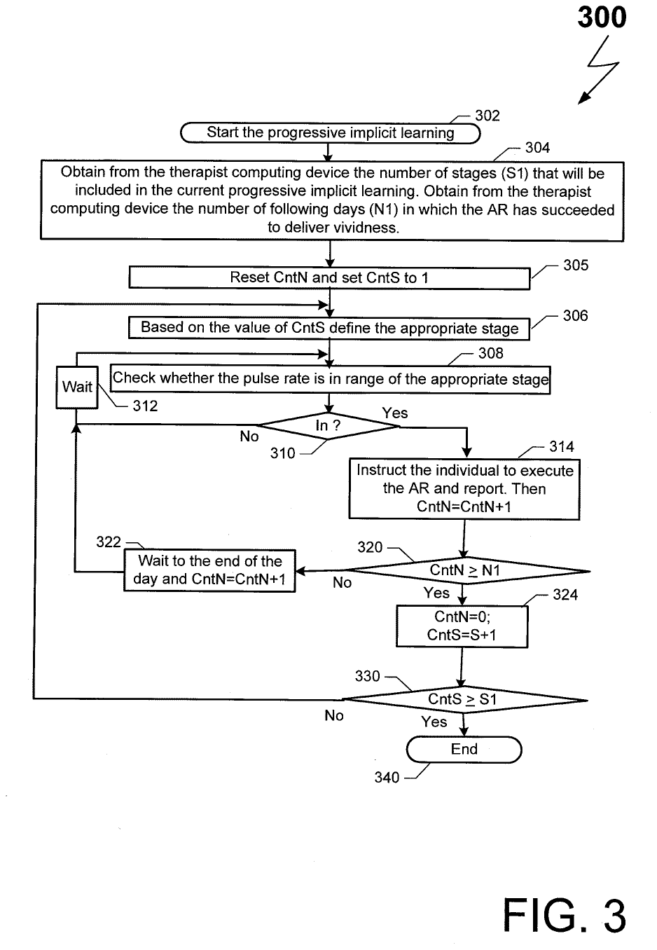

Referring now to FIG. 3 that schematically illustrates a flowchart showing relevant processes of an application 300 that can be used for progressive implicit learning of an individual. Application 300 can be executed by the IBDRC 120 (FIG. 1) after the therapist determines that the individual is ready to use the AR.

At block 304 the application can request to obtain from the therapist computing device 150 (FIG. 1) the number of stages S1 to be included in the current progressive implicit learning and the number of following days (N1) that the AR succeeds to deliver vividness. An example embodiment of the disclosed technique may use 3 stages (S1=3) and the number of the following days can be 4 days (N1=4). Other example embodiments may use other values for S1 and N1.

Example embodiments, in which the pulse rate is used as the biological parameter, each stage can be defined as an interval of pulse rates. The first stage can start with a low pulse rate, 60 PPM for example. The second stage can be started with a pulse rate of 75 PPM for example and the last stage can start with pulse rate of 90 PPM for example. Other embodiments of the disclosed technique may use other number of stages and/or use other values of pulse rate as the interval of each stage, or may use other biological parameter such as but not limited to GSR, HRV, Stress, etc. Further, other example embodiments may use a combination of two or more biological parameters. A combination of blood pressure and pulse rate can be used in order to define the borders between stages, for example.

At block 305 counter CntN can be reset and counter CntS can be set to 1. Then a loop, per each stage, can be initiated at block 306 and be terminated in block 330. In block 306 based on the value of CntS the appropriate stage is defined. The stage in which the individual is currently involved. For stage 1 at block 308 application 300 can check whether the pulse rate, or a combination of two or more bio-parameters, of the individual is in the interval of stage 1. If 310 the pulse rate is not in the interval of stage 1, then application 300 may wait 312 for a period of D1. The value of D1 can be in the range of few tens of minutes, 10 to 30 minutes for example. If 310 the pulse rate is in the interval of stage 1, then application 300 may instruct the individual 314 to execute the AR and to report his feelings. In some embodiments, application 300 may check the HRV monitor in order to get an objective indication of the client status and his progress in the treatment. Then, CntN can be incremented by one.

At block 320 a decision is made whether the value of CntN is equal or greater than the value of N1. If 320 the value is not greater, then the application 300 may wait 322 to the end of the day for incrementing CntN by 1 and return to block 312.

If the value of CntN is equal or greater than N1, then at block 324 CntN can be reset and CntS can be incremented by 1 and a decision is made 330 whether the value of CntS is greater than S1 (3 for example), which means that the final stage (3, for example) of the progressive implicit learning is terminated and the application 300 can be terminated 340. If 330 the value of CntS is smaller than S1, then application 300 can return to block 306 for executing the next stage of the progressive implicit learning.

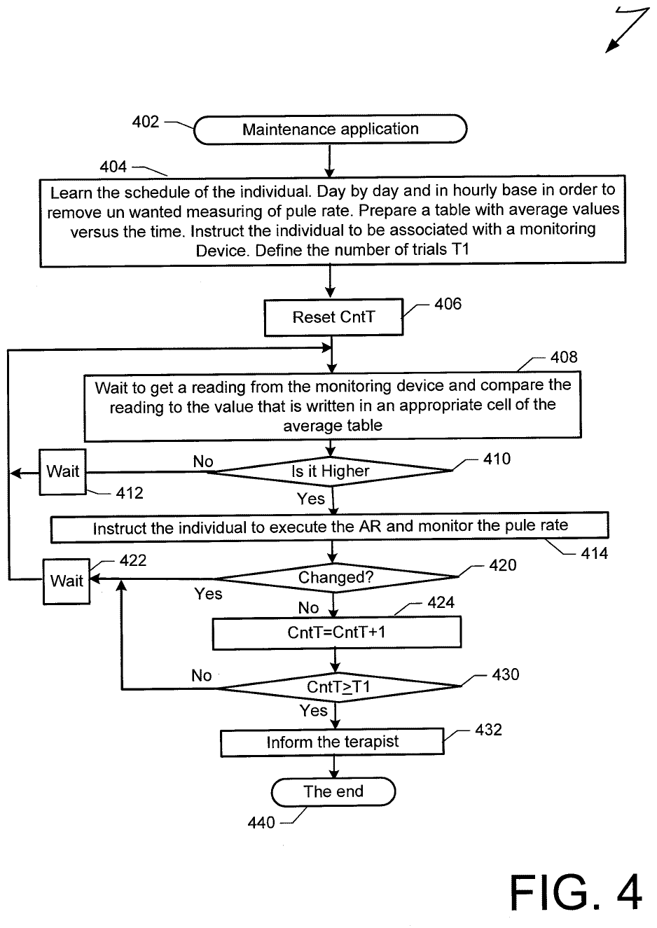

After finishing the progressive implicit learning that is disclosed above, the individual is requested to download to the individual's smart phone 120 (FIG. 1) an example of a maintenance application. An example of the maintenance application 400 is described in FIG. 4. After initiation 402, the individual is requested to wear 404 a monitoring device. A not limiting example of a monitoring device can be a device that is configured to measure the pulse rate of the individual, ring “R10” for example. Other example of a monitoring device can be smart-watch such as but not limited to “VIVOACTIVE 5”. Then, at block 404 the application may learn the weekly schedule of the individual. The schedule may comprise the rising hour, eating hours, working hours, exercising hours, etc.

Some example embodiment of application 400 may prepare a table 404 per each day. Each row can represent an hour in that day the first column can be associated with the type of activity of that individual, the second column can indicate the low pulse rate that was measured during that hour and the 3^{rd} column can be associated with the highest pulse rate that was measured during that hour. Another column can indicate the type of activity (sleeping, eating, running, at work etc.) and in the last column the individual can indicate the level of vividness that the individual felt at the moment of reporting. In parallel the pulse rate can be recorded too in the table. The monitoring device can be configured to send periodically its reading toward the IBDRC 120 (FIG. 1). An example period can be in the range of few tens of seconds, 30 seconds for example.

The learning process 404 can take few weeks, two to five weeks, for example. At the end of process 404 an average table can be prepared per each day, wherein each cell of that table is the average of the pulse rate that is written in a relevant cell in each table. The average table can be stored in the appropriate section of IDB 114a-m. In addition at block 404 application 400 may define the number of trials (T1) that individual will fail to reach vividness before informing the therapist.

After learning the schedule of the individual, process 400 may 406 reset the value of counter CntT that counts the number of trials. At block 408 process 400 may wait to get the reading from the monitoring device. In some example embodiment the reading can be the pulse rate that is measured and be compared to the pulse rate that is written in the appropriate cell of the average table. The appropriate cell can be a cell in the table that is related to the day and the hour of the current moment. At block 408 the value of the current measured pulse rate is compared to pulse rate that is written in the appropriate cell in the table and a decision is made 410 whether the current measured pulse rate is higher than the one that is written in the table.

If 410 the current pulse rate is not higher, then process 400 may wait 412 for a period of few minutes, 1-7 minutes for example, and return after 5 minutes for example, to block 406. If 410 the pulse rate is higher, then the application can send an indication 414 to the individual, requesting the individual to execute the AR and to monitor the pulse rate. At block 420 a decision is made whether the pulse rate is changed, which indicates that the current state of the brain disruption is improved. If 420 yes, then process 400 may wait 422 for a period of few minutes, 1-7 minutes for example, and return after 5 minutes for example, to block 406. If 420 the pulse rate remains high, then the application may increment 424 counter (CntT) by one and a decision is made 430 whether the value of CntT is greater or equal to R1.

Other example embodiments may use a combination of two or more biological parameters. A combination of blood pressure and pulse rate can be used in order to distinguish between high-positive-emotions and high-negative-emotions, for example

If 430, CntT is not greater or equal to R1 then application 400 may return to block 422. If CntT is greater or equal to R1, then application 400 may send 432 a message to the therapist-device 150 (FIG. 1) informing the therapist that the AR did not help and process 400 can be terminated 440. The therapist may change few parameters and may ask the individual to return to block 406. The changed parameters may include the pulse rate that is used to invoke the AR, for example. In some example embodiments of process 400, after informing the therapist process 400 can be terminated 440.

In some cases the therapist may determine to return to the progressive learning (application 300 FIG. 3) and may use other type of auto biography positive memory in order to bring a PEEM into the present, etc.

In this disclosure the words “module,” “device,” “component,” “element” and “unit” may be used interchangeably. Anything designated module may be a stand-alone module or a specialized module. A module may be modular or have modular aspects allowing it to be easily removed and replaced with another similar module. In this disclosure and the claims the words “process,” “application”and “method,”may be used interchangeably.

In the description and claims of the present disclosure, “comprise,” “include,” “have,” and conjugates thereof are used to indicate that the object or objects of the verb are not necessarily a complete listing of members, components, elements, or parts of the subject or subjects of the verb.

It will be appreciated that the above-described apparatus, systems and methods may be varied in many ways, including, changing the order of steps, and the exact implementation used. The described embodiments include different features, not all of which are required in all embodiments of the present disclosure. Moreover, some embodiments of the present disclosure use only some of the features or possible combinations of the features. Different combinations of features noted in the described embodiments will occur to a person skilled in the art. Furthermore, some embodiments of the present disclosure may be implemented by combination of features and elements that have been described in association to different embodiments along the discloser.

It is to be understood that the above description is intended to be illustrative, and not restrictive. For example, the above-described embodiments may be used in combination with each other. Many other embodiments will be apparent to those of skill in the art upon reviewing the above description.

The scope of the invention therefore should be determined with reference to the appended claims, along with the full scope of equivalents to which such claims are entitled. In the appended claims, the terms “including” and “in which” are used as the plain-English equivalents of the respective terms “comprising” and “wherein.”

What is claimed is:

1: A non-transitory computer readable storage device comprising executable instructions that when executed cause a processor at an Individual's-Brain-Disruption-Release Controller (IBDRC):
to obtain, for an individual with brain disruption, two or more intervals of a biological-parameter, wherein each interval is associated with a stage of a progressive-implicit-learning;
to determine that a current reading from a monitoring device, which is associated with the IBDRC and is configured to monitor the biological-parameter, is in the interval of a current stage of the progressive-implicit-learning; and
to instruct the individual, who is associated with the IBDRC, to move toward a following stage.

2: The non-transitory computer readable storage device of claim 1, wherein the following stage comprising instructions that when executed cause the processor at the IBDRC to instruct the individual to execute an anchoring-ritual (AR).

3: The non-transitory computer readable storage device of claim 1, wherein the brain disruption is chronical-psychological-stress.

4: The non-transitory computer readable storage device of claim 1, wherein the brain disruption is a body-mind-disorder.

5: The non-transitory computer readable storage device of claim 1, wherein the biological-parameter is heart pulse rate.

6: The non-transitory computer readable storage device of claim 2, wherein the AR comprises associating a finger with a thumb of the right hand of the individual.

7: The non-transitory computer readable storage device of claim 2, wherein the executing AR leads the individual toward vividness.

8: The non-transitory computer readable storage device of claim 1, wherein the monitoring device is configured to monitor the heart pulse rate of the individual.

9: The non-transitory computer readable storage device of claim 8, wherein the monitoring device is a ring.

10: The non-transitory computer readable storage device of claim 1, further comprising instructions that when executed cause the processor at the IBDRC to play a recorded file, RecF1, which comprises autobiographic-positive-memory (APM) of the individual.

11: The non-transitory computer readable storage device of claim 10, wherein the RecF1 further comprises music that the individual likes.

12: The non-transitory computer readable storage device of claim 10, wherein describing the APM in the RecF1, is done by a therapist.

13: The non-transitory computer readable storage device of claim 12, wherein RecF1 is stored in a server that is associated with the therapist.

14: The non-transitory computer readable storage device of claim 1, wherein the first stage of the progressive-implicit-learning is associated with a first interval of the biological-parameter.

15: The non-transitory computer readable storage device of claim 1, further comprising instructions that when executed cause the processor at the IBDRC:
to learn a schedule of the individual; and
to determine, based on the schedule of the individual, that a current reading from the monitoring device is not related to the brain disruption.

16: The non-transitory computer readable storage device of claim 1, further comprising instructions that when executed cause the processor at the IBDRC to verify that executing the AR improves the current state of the individual.

17: A system for treating Brain-Disruption of an individual, the system comprising:
a therapist-computing-device (TCD) that is configured to prompt a therapist:
i. during a meeting with an individual to lead an individual, who suffers from a brain disruption, toward vividness by using autobiographic-positive-memory (APM) of the individual; to prepare a first vocal recorded file RecF1; and to ask the individual to play the RecF1 ‘N1’ times a day for a period of ‘M1’ days;
ii. during another meeting, to persuade the individual that using an anchoring-ritual (AR) brings a past emotional experience memory (PEEM) into the present for releasing the brain disruption; to prepare a second vocal recorded file RecF2. and to ask the individual to play it ‘N2’ times a day for a period of ‘M2’ days;
iii. to plan a progressive-implicit-learning of the individual, wherein the progressive-implicit-learning comprises two or more stages wherein each stage is associated with a different level of one or more biological parameters; and to prepare two or more vocal recorded files, one per each stage;
and
an Individual's-Brain-Disruption-Release-System (IBDRS) comprising an Individual's Brain-Disruption-Release-Controller (IBDRC) and a monitoring device that is configured to monitor the one or more biological parameter of the individual that is related to the brain disruption.

18: The system of claim 17, wherein the brain disruption is chronical-psychological-stress.

19: The system of claim 17, wherein the IBDRS further comprising real time neuro waves monitoring device.

20: The system of claim 17, wherein N1 and N2 are integer numbers between two to five.

21: The system of claim 17, wherein M1 and M2 are integer numbers between five to fifteen.

22: The system of claim 17, wherein the biological parameter is heart pulse rate.

23: The system of claim 17, wherein the brain disruption is a body-mind-disorder.

24: The system of claim 17, wherein the monitoring device is configured to monitor heart-rate-variations (HRV).

25: The non-transitory computer readable storage device of claim 1, wherein the monitoring device is configured to monitor heart-rate-variations (HRV).
